(19) United States
(12) Patent Application Publication
(10) Pub. No.: US 2011/0282437 A1
(43) Pub. Date: Nov. 17, 2011

(54) ENDOPROSTHESIS

(75) Inventors: Robert W. Warner, Woodbury, MN (US); Steve Kangas, Woodbury, MN (US); Liliana Atanasoska, Edina, MN (US); Joseph Thomas Ippoliti, Woodbury, MN (US); Scott R. Schewe, Eden Prairie, MN (US)

(73) Assignee: BOSTON SCIENTIFIC SCIMED, INC., Maple Grove, MN (US)

(21) Appl. No.: 13/105,488

(22) Filed: May 11, 2011

Related U.S. Application Data
(60) Provisional application No. 61/334,691, filed on May 14, 2010.

(51) Int. Cl. A61F 2/82

(52) U.S. Cl. 623/1.44

(57) ABSTRACT

In embodiments, a stent comprises a biodegradable polymer functionalized with an adhesion-enhancing amino acid.

### CROSS-REFERENCE TO RELATED APPLICATION

This application claims priority under 35 USC §119(e) to U.S. Provisional Patent Application Ser. No. 61/334,691, filed on May 14, 2010, the entire contents of which are hereby incorporated by reference.

### TECHNICAL FIELD

The present invention relates to endoprosthesis, and more particularly to stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

It is sometimes desirable for an implanted endoprosthesis to erode over time within the passageway. For example, a fully erodible endoprosthesis does not remain as a permanent object in the body, which may help the passageway recover to is natural condition. Erodible endoprostheses can be formed from, e.g., an erodible polymeric material such as polylactic acid, and/or from an erodible metallic material such as magnesium, iron, or an alloy thereof.

### SUMMARY

The present invention is directed to an endoprosthesis, such as, for example, a biodegradable stent.

In one aspect, the invention features a stent including a first layer including a biodegradable polymer functionalized with an adhesion enhancing amino acid.

In another aspect, the invention features a stent, including a stent body, a first layer over the stent body including a biodegradable polymer functionalized with an adhesion enhancing amino acid, a second layer over the first layer including a second biodegradable polymer functionalized with an adhesion enhancing amino acid; and a third layer over the second layer, the third layer including a drug-eluting polymer.

Embodiments of the battery may include one or more of the following features.

The first layer can be on a stent body (e.g., a metal stent body). The stent body can include a stainless steel, a polymer, a ceramic, a metal, a metal alloy, a metal oxide, a metal nitride, and/or mixtures thereof. In some embodiments, the first layer is directly on a stent body (e.g., a metal stent body). The amino acid can include DOPA. The DOPA can be polymerized. The polymer can include PLA, PLGA, PLLA, polydioxanone, chitosan, polycaprolactone, blends thereof, or copolymers thereof. The DOPA can be oxidized. The oxidized DOPA can include a quinone or semiquinone moiety. The DOPA can be nonoxidized.

The stent can further include a second layer over the first layer, the second layer can include a second biodegradable polymer functionalized with an adhesion enhancing amino acid. The first and second biodegradable polymers can be the same or different. The stent can further include a third layer over the first and second layers, wherein the third layer includes a drug-eluting polymer. The first or the second layer can include, independently, an oxidized DOPA and/or a nonoxidized DOPA. The first and the second layers can include, independently, a metal such as iron, magnesium, and/or oxides thereof. The first layer can be only on the abluminal surface of the stent body. The first layer can further include a therapeutic agent. In some embodiments, the first layer, the second layer, and the third layer each optionally include, independently, a therapeutic agent.

Embodiments may include one or more of the following advantages. A stent is provided with advantageous drug delivery characteristics, mechanical properties and biodegradability. The stent can include a biodegradable polymer including an amino acid component that enhances adhesion to a stent body. In particular embodiments, 3,4-dihydroxyphenylalanine (DOPA) having L or R stereochemistry, preferably L, an amino acid found in mussel adhesive proteins, is conjugated to biodegradable polymers such as PLGA, PLLA, chitosan, polysaccharides, and/or blends thereof. The high adhesion of the polymer can allow for direct use of the polymer on a stent body that has not been modified with, for example, layers of ceramic, silanes or on a stent body that has not been roughened. For example, the polymer can be applied directly to polished metal surfaces, e.g., stainless steel. The high adhesion also permits use of the polymer as non-conformal coating, i.e., coating on select surfaces of the stent, such as the abluminal surface. In some embodiments, the biodegradable polymer can be provided as a layer over a stent body, e.g., made of a metal, such as a biodegradable metal, or made of an erodible material, such as an erodible polymer.

The details of one or more embodiments are set forth in the accompanying drawings and the description below. Other features, objects, and advantages will be apparent from the description and drawings, and from the claims.

### DESCRIPTION OF DRAWINGS

FIGS. 1A-1C are sequential, longitudinal cross-sectional views, illustrating delivery of an endoprosthesis in a collapsed state, expansion of the endoprosthesis, and the deployment of the endoprosthesis in a body lumen;

FIG. 2A is a perspective view of an embodiment of a stent;

FIG. 2B is magnified view of a section of the stent in FIG. 2A; and

FIG. 3 is a cross-sectional view of a section of a stent strut.

### DETAILED DESCRIPTION

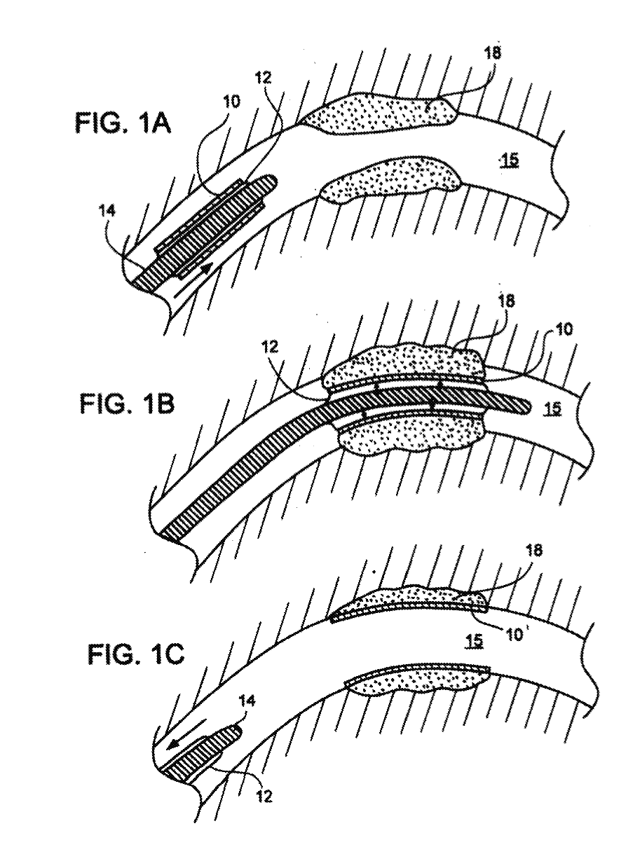

Referring to FIGS. 1A-1C, a stent 20 is placed over a balloon 12 carried near a distal end of a catheter 14, and is directed through the lumen 16 (FIG. 1A) until the portion carrying the balloon and stent reaches the region of an occlusion 18. The stent 20 is then radially expanded, e.g., by inflating the balloon 12, and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C).

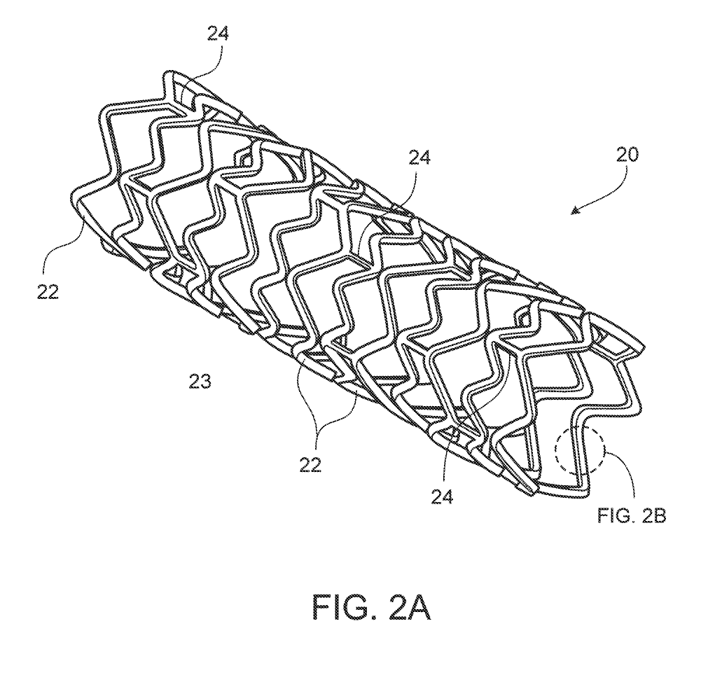

Referring to FIG. 2A, an expandable stent 20 can have a stent body having the form of a tubular member defined by a plurality of bands 22 and a plurality of connectors 24 that extend between and connect adjacent bands. During use, bands 22 can be expanded from an initial, smaller diameter to a larger diameter to contact stent 20 against a wall of a vessel, thereby maintaining the patency of the vessel. Connectors 24 can provide stent 20 with flexibility and conformability that allow the stent to adapt to the contours of the vessel. One or more bands 22 form acute angles 23. The angle 23 increases upon expansion of the stent. Stent body 20, bands 22 and connectors 24 can have a luminal surface 26, an abluminal surface 28, and a sidewall surface 29. In embodiments, the bands and/or connectors, have a width, W, and a thickness, T, of about 50 to 150 microns.

Referring to FIG. 2B, the stent body 30 carries a coating 32 on the stent body surface. The coating can include a therapeutic agent which is released to, for example, inhibit restenosis. The coating can have a thickness, Tc. In embodiments, the coating can carry substantial loading of drug and exhibit desirable agent release profiles, such as zero order release. As a result, in some embodiments, the thickness Tc can be thin, which provides for a low overall profile, good adhesion to the stent body surface, and less foreign material introduced in the body. For example, in embodiments, the thickness Tc of the coating is about 10 μm or less, e.g., five μm or less or one μm or less. In some embodiments, the thickness Tc of the coating is about 0.1 μm or more (e.g., 0.5 μm or more, one μm or more, five μm or more, 10 μm or more, 25 μm or more, or 50 μm or more) and/or 100 μm or less (e.g., 50 μm or less, 25 μm or less, 10 μm or less, five μm or less, one μm or less, or 0.5 μm or less). In particular embodiments, the coating is biodegradable. In FIG. 2B, the coating is illustrated on the abluminal surface. In embodiments, one or more coatings may instead or in addition be on the luminal and/or side wall surfaces. In particular embodiments, the coating is adhered directly to the body of the stent, which is formed, e.g., of a metal such as stainless steel, or of a biodegradable material such as a biodegradable polymer and/or biodegradable metal.

In some embodiments, coating 32 includes a biodegradable polymer that has been modified by conjugation of amino acids (e.g., 3,4-dihydroxy-phenylalanine “DOPA”) to generate an adhesive amino acid-functionalized polymer (e.g., DOPA-functionalized polymer) that enhances adhesion of the polymeric coating to the stent body. In particular embodiments, the biodegradable polymer is a polylactic acid (PLA), poly(lactic-co-glycolic acid) (PLGA), poly-L-lactide (PLLA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly(alpha-hydroxy acid), their copolymers, blends, and combinations thereof, and the amino acid is 3,4-dihydroxy-L-phenylalanine (DOPA). Without wishing to be bound by theory, it is believed that DOPA is a prominent amino acid found in mussel adhesive proteins that is responsible for mussels' ability to tenaciously bind to various substrates under water. Conjugating this amino acid to a biodegradable polymer can provide adhesive properties to the polymer and the amino acid can act as an adhesion promoter to couple drug-eluting polymers to medical devices. In some embodiments, the adhesive amino acids can include polyamino acids, such as a polyamino acid including a first amino acid selected from glutamate, asparagine, aspartate, and glutamine; a second amino acid selected from lysine and arginine; and a third amino acid selected from cysteine, methionine, serine, threonine, glycine, alanine, valine, leucine, and isoleucine. Other adhesive polyamino acids are described, for example, in U.S. Pat. No. 5,733,868, herein incorporated by reference in its entirety.

In some embodiments, the adhesive amino acid-functionalized polymer can have an adhesive amino acid content of more than 10% (e.g., more than 20%, more than 30%, or more than 40%) adhesive amino acid and/or less than 10% (e.g., less than 5%, less than 2%, or less than 1%) adhesive amino acid relative to a total monomer content (including the adhesive amino acid). In some embodiments, the adhesive amino acid-functionalized polymer can have an adhesive amino acid content of more than 0.1% (e.g., more than 1%, more than 5%, or more than 10%) adhesive amino acid and/or less than 60% (e.g., less than 40%, less than 30%, less than 20%, or less than 10%) adhesive amino acid relative to a total monomer content (including the adhesive amino acid). For example, a polylactide include 97 repeating lactide monomers and one adhesive polyamino acid molecule of three component amino acids would have an adhesive amino acid content of three percent.

In some embodiments, when the DOPA-functionalized polymer includes a polymer of lactic acid, glycolic acid, and a free amino acid DOPA, hydrolysis of the polymer can produce biocompatible degradation products including DOPA, glycolic acid, and lactic acid. The free amino acid DOPA is an oxidation product of tyrosine and a normal metabolite, and is involved in pathways of dopamine and melanin production.

In some embodiments, coating 32 can include a blend including DOPA-functionalized polymer. The DOPA-functionalized polymer can be blended with unmodified biodegradable polymers (e.g., polylactic acid, polyglycolic acid, or polylactic acid-polyglycolic acid copolymer, or other biodegradable polymers) to convey adhesive properties to the blend. For example, in some embodiments, a blend can include 0.1% or more (e.g., 1% or more, 5% or more, 10% or more, 20% or more, 30% or more, or 40% or more) and/or 10% or less (e.g., 40% or less, 30% or less, 20% or less, 10% or less, 5% or less, or 1% or less) by weight of the DOPA-functionalized polymer.

Coating 32 can be tailored to have differing levels of tackiness by altering the ratio of DOPA to total monomer or polymer components. For example, a blend having a greater proportion of a DOPA-functionalized polymer can have increased tackiness compared to a blend having less DOPA-functionalized polymer. A polymeric material, whether a DOPA-functionalized polymer, or a blend containing DOPA-functionalized polymer, having a higher ratio of DOPA to total monomer can be more tacky than a polymeric material having a lower ratio of DOPA to total monomer. In the case of a DOPA-functionalized block copolymer containing a DOPA-functionalized block and a non-DOPA-functionalized block, the tackiness can also be modified by altering the length of a non-DOPA-containing block component within the block copolymer. Tackiness can also be altered depending on the ratio of DOPA-functionalized block to non-DOPA-functionalized block, where the greater the non-DOPA-functionalized block content, the less tacky the resulting block copolymer. Tackiness for a DOPA-functionalized polymer can be measured with a Tack Tester, available, for example, from Testing Machines Inc., Ronkonkoma, N.Y. In some embodiments, when coated on a surface, the tackiness of a DOPA-functionalized polymer can be measured by atomic force microscopy (AFM) force measurement techniques, as described, for example, in Catron et al., Enhancement of Poly(ethylene glycol) Mucoadsorption by Biomimetic End Group Functionalization, Biointerphases, (2006), 1(4), December 2006. In some embodiments, adhesive properties of a coating of DOPA-functionalized polymer can also be assessed using an axisymmetric indentation method, as described, for example, in Guvendiren M. et al., Self-Assembly and Adhesion of DOPA-Modified Methacrylic Triblock Hydrogels, Biomacromolecules (2008) 9, 122-128.

In some embodiments, coating 32 can include plasticizers. The plasticizers can help tune the adhesive and kinetic drug release properties of a polymer. For example, the polymer can include one percent or more (e.g., 1% or more, 5% or more, 10% or more, or 15% or more) and/or 20% or less (e.g., 15% or less, 10% or less, 5% or less, or 1% or less) by weight of the plasticizer. Examples of plasticizers for polymers include, for example, polyethylene glycol, triethyl citrate, diethylphthalate, fatty acids (e.g., palmitic acid, oleic acid), and monomer constituents of the polymers. Plasticizers are described, for example, in Yahya et al., Ionic Conduction Model in Salted Chitosan Membranes Plasticized with Fatty Acid, Journal of Applied Sciences (2006), 6(6), 1287-91; El-Bagory et al., Effects of Sphere Size, Polymer to Drug Ratio and Plasticizer Concentration on the Release of Theophylline from Ethylcellulose Microspheres, Saudi Pharmaceutical Journal, (2007) 15(3-4), 213-17; and in Dias et al., The Influence of Plasticizer Type and Level on Drug Release from Ethylcellulose Barrier Membrane Multiparticulates, available from the Internet at www.colorcon.com.cn.

In some embodiments, as discussed above, the DOPA-functionalized polymer serves as an adhesion promoter to couple polymer and/or metal layers to a stent surface. As an example, the DOPA-functionalized polymer can be used as a tie layer between a stent surface and a second layer of polymer or metal-containing layer (e.g., a drug delivery polymer layer, or a drug delivery metal layer). For example, the DOPA-functionalized polymer can be first applied to the surface of a stent, and a second layer of a drug-containing polymer can be coated on top of the DOPA-functionalized polymer tie layer. By using a DOPA-functionalized polymer as a tie layer, it is believed that the drug delivery characteristics of the drug-containing polymer can function independently of the DOPA content in the coatings.

The DOPA-functionalized polymer can also serve as a drug delivery vehicle for controlled release of anti-restenotics, antibiotics, anti-inflammatory and other biologically active agents, with the benefit of tunable biodegradation and tunable kinetic drug release. In general, the biodegradation and drug release profile of the DOPA-functionalized polymer or drug-eluting polymer can be tuned, for example, by varying the molecular weight of a polymer, the copolymerization ratio of various block components of a block copolymer, the monomer ratio of a multi-component polymer (e.g., lactide to glycolide ratio for a PLGA polymer), the drug to polymer ratio, the plasticizer content, and/or the coating thicknesses. A drug can be incorporated by preparing a solution of the polymer and drug, followed by application of the solution to a stent.

Inorganic-Organic Hybrids Coatings

The oxidation state of the DOPA molecule can be used to enhance adhesion to a variety of organic and inorganic substrates. Without wishing to be bound by theory, it is believed that oxidation of DOPA (e.g., to a DOPA-quinone and/or a DOPA-semiquinone) increases adhesion to organic substrates whereas unoxidized DOPA tends to increase adhesion to inorganic substrates. The oxidation of DOPA to quinone DOPA and the reduction of quinone DOPA to DOPA are illustrated in Scheme 1.

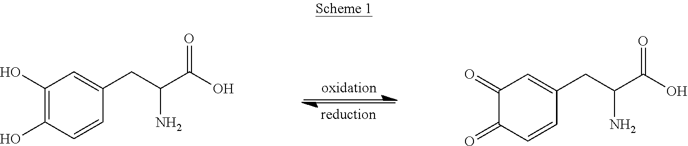

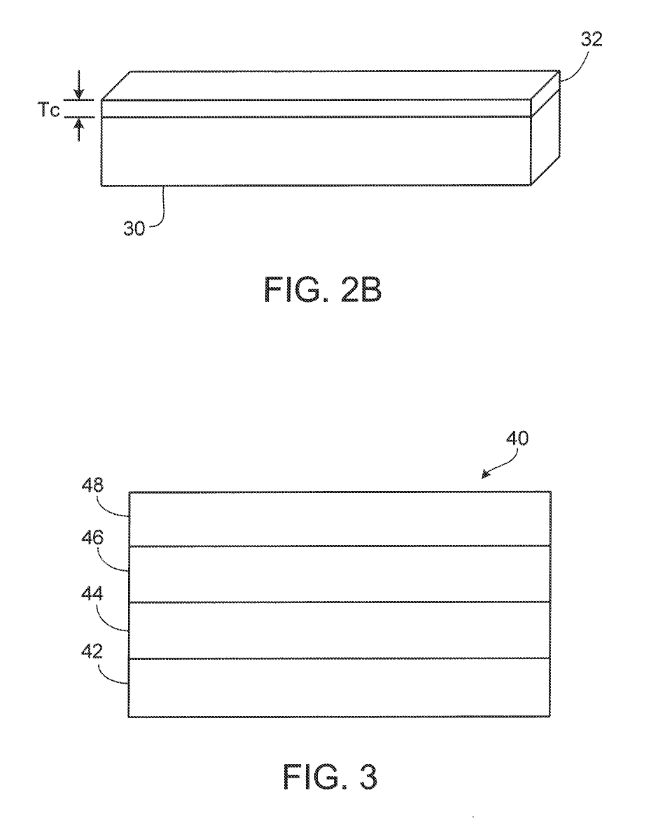

In some embodiments, an inorganic-organic hybrid material is modified with DOPA. For example, a biodegradable polymer layer can include biodegradable metals (e.g., Fe or Mg) and/or their oxides, together with a DOPA-functionalized biodegradable polymer. Biodegradable organic-inorganic hybrid materials can be made from DOPA-containing co-polypeptides, proteins, an/or polymers, with metals and/or oxides of metals (e.g., magnesium oxides, iron oxides). DOPA-containing polypeptides are described, for example, in Deming T. J., “Synthetic polypeptides for biomedical applications”, Prog. Polym. Sci. (2007) 32, 858-875. In some embodiments, the inorganic phase is a biocompatible material such as Ca, Fe or Mg phosphates. Referring to FIG. 3, a stent strut 40 can include two or more layers made from reduced or oxidize DOPA-containing co-polypeptides. The layers can function as an abluminal adhesion tie layer between a drug-eluting polymer (e.g., bioerodible polymers and/or biostable polymers) over-coat and a stent body surface. The DOPA-containing layers can serve as an alloy-specific corrosion inhibitor for metallic stents (e.g., PERSS, Co—Cr or 316L stainless steel stents). The mechanism of adhesion of DOPA and DOPA-quinone to inorganic and organic surfaces is described, for example, in Lee et al., “Single-Molecule Mechanics of Mussel Adhesion”, PNAS, (2006), 103(35), 12999-13003. In some embodiments, the drug-eluting polymer over-coat can also be functionalized with DOPA-containing polymers, as disclosed supra.

Referring to FIG. 3, a stent 40 can have a first layer 44 of un-oxidized (reduced) state of DOPA peptides including hydroxy-DOPA, which can adhere to an oxide or iron-containing stent surface 42 via reversible coordination bonds, as illustrated in Scheme 2 below.

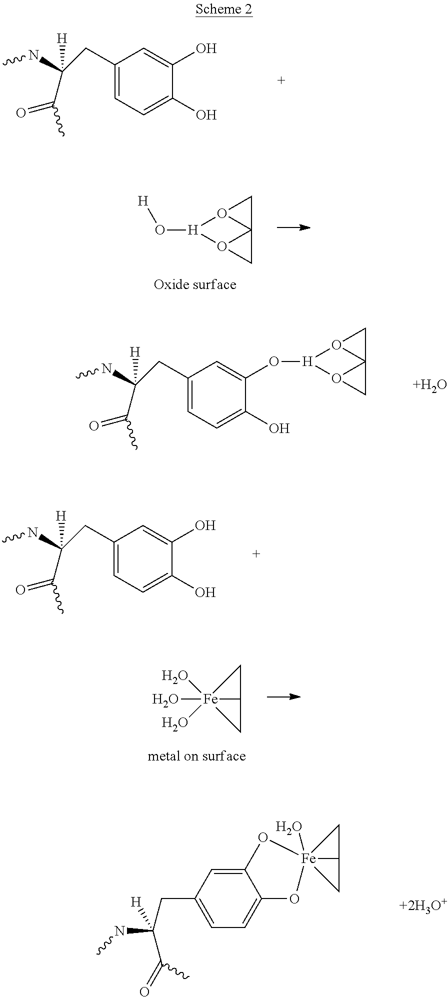

In some embodiments, referring to Scheme 3, it is believed that Fe³⁺ ion can chelate to DOPA to optimize adhesive strength, to increase water resistance, and/or to reduce swelling of the DOPA-containing adhesive polymer in an aqueous environment.

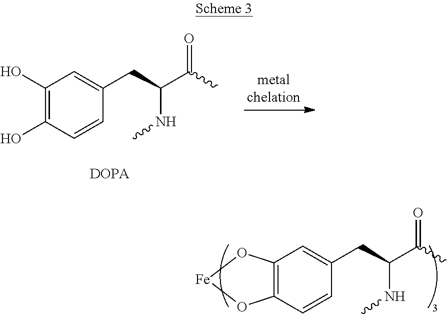

Referring to FIG. 3, stent 40 can have a second layer 46 of oxidized state of DOPA peptides including DOPA-quinone that can adhere to organic polymer surfaces (e.g., PLGA and/or PLA) via covalent bond formation. Layer 46 can enhance adhesion of an overlaying drug-eluting layer 48 including an organic polymer while also adhering to an organic polymer present in layer 44. A stent including a drug eluting layer over DOPA-functionalized tie layers can promote endothelial cell growth over an implanted stent.

In some embodiments, DOPA-containing tie layers can be positioned between a drug-eluting metallic (e.g., a drug-eluting bioerodible metallic, a drug-eluting bioerodible porous metallic) over-coat and a stent body surface.

The DOPA-containing tie layers (e.g., 44 and 46) can include additives. In some embodiments, magnesium can be added to layer 46 to form a slightly alkaline environment to convert/maintain DOPA in its quinone state for improved adhesion of drug-eluting polymeric layer 48. In some embodiments, Fe is added to provide ferric ions to provide chelation with DOPA-containing polymers, (e.g., to enhance crosslinking with the DOPA-containing polymers), to provide increased adhesion strength of a DOPA-containing polymers to the underlying stent surface 42, and to provide greater corrosion protection of the underlying stent surface. Addition of biodegradable Fe and Mg metal additives with L-DOPA polymers can tune the adhesion performance of the DOPA-containing tie layers (e.g., 44 and 46) by creating a dynamic oxidizing environment of alkaline pH and ferric ions chelation by the DOPA polymers.

In some embodiments, to achieve water-resistant adhesion, the DOPA segments of the polymeric conjugates are un-oxidized or “quinone tanned” by non-enzymatic cross-linking. Oxidation and reduction of DOPA are described, for example, in Waite J. H., Adhesion à la Moule, (2002), Integr. Comp. Biol., 42:1172-1180; Doraiswamy, A. et al., Matrix-assisted pulsed-laser evaporation of DOPA-modified poly(ethylene glycol) thin films, J. Adhesion Sci. Technol., (2007) 21(3-4), 287-299; Guvendiren M. et al., Self-Assembly and Adhesion of DOPA-Modified Methacrylic Triblock Hydrogels, Biomacromolecules (2008) 9, 122-128.

In some embodiments, the DOPA-functionalized polymer can be spun into nano/micro fibers and serve as a component of a stentless therapeutic device or a vascular scaffolding device component either as an adhesion promoter to tissue, or as a drug elution media, or both.

Coatings including DOPA-functionalized polymers can be made by dissolving the DOPA-functionalized polymers in a suitable solvent to form a solution or a suspension, and applying the solution or suspension to a stent by dipping, spraying, roll-coating, or other coating techniques. Adhesion of coatings of DOPA-functionalized polymer to the stent can occur as the solvent evaporates.

### Synthesis of DOPA-Functionalized Polymers

In some embodiments, the monomers of a biodegradable polymer (e.g., the lactide of lactic acid (LA lactide) of PLA) contain no functionalized side chains (“unfunctionalized monomer”). In such a scenario, a monomer with a functionalized side chain is copolymerized with the unfunctionalized monomer. As an example, for PLA, a monomer with a functionalized (e.g., a reactive functionality-containing) side chain can be a protected lactone of serine, which contains a functionalizable amino side chain. As another example, for PLA, a monomer with a functionalized side chain can be a functionalized lactide, which can have similar polymerization rates as the LA lactide used to make PLA. DOPA can be conjugated to a functionalized monomer prior to ring opening polymerization (ROP) with LA lactide to give a polylactic acid copolymer. Alternatively, DOPA can be conjugated a functionalized copolymer (e.g., functionalized PLA) after the ROP process. Several organic catalysts exist to initiate the ROP process so that subsequent removal processes of conventional organo-metallic catalysts (e.g., tin(II)2-ethylhexanoate) is no longer necessary. For example, the organic catalysts can include N-imidazolium carbenes, thiazaolium carbenes, 1,8-diazabicyclo[5.4.0]-undec-7-ene (DBU), N-methyl-1,5,7-triazabicyclo[4.4.0]dec-5-ene (MTBD), and/or triazabicyclo[4.4.0]dec-5-ene (MTBD).

Certain methods for the preparation of DOPA-containing polymers of the present invention include, but are not limited to, those described in Schemes 4-6 set forth in this section of the specification. The DOPA-functionalized polymers can be prepared according to a variety of synthetic manipulations, all of which would be familiar to one skilled in the art of synthetic organic chemistry. While Schemes 4-6 illustrate polymers that are fully reacted with DOPA, it is understood that polymers can also be partially substituted with DOPA such that some reactive functional groups on the polymers are unreacted. For example, more than 20% (more than 50%, more than 70%, more than 90%) and/or less than 99% (e.g., less than 90%, less than 70%, less than 50%) of the reactive functional groups on the polymers (e.g., NH₂, OH, or other nucleophiles) can remain unreacted with DOPA. Further, while DOPA is illustrated in Schemes 4-6, it is understood that polymers including other adhesive amino acids, including polyamino acids, can be prepared in a similar manner, according to a variety of synthetic manipulations familiar to one skilled in the art of synthetic organic chemistry.

For example, referring to Scheme 4, 3,6-dimethyl-1,4-dioxane-2,5-dione (I) is polymerized with a protected 3-aminooxetan-2-one (II), in the presence of a ROP catalyst. The resulting polymer (III) can be a block copolymer, or a random copolymer. In the case of a block copolymer, variable m can be, for example, 5 or more (e.g., 10 or more, 15 or more, 20 or more, 25 or more) and/or 30 or less (e.g., 25 or less, 20 or less, 15 or less, or 10 or less). Variable n can be, for example, 1 or more (e.g., 3 or more, 5 or more, or 7 or more) and/or 10 or less (e.g., 7 or less, 5 or less, or 3 or less).

After formation of the protected polymer (III), deprotection is carried out, and the polymer is coupled to a protected DOPA molecule (V) to afford a DOPA-functionalized polymer (VI).

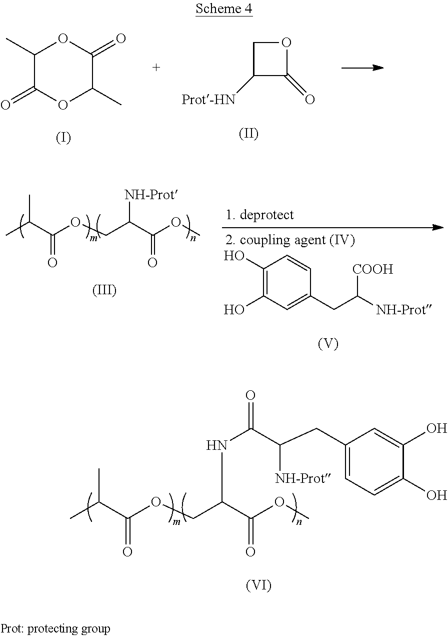

In some embodiments, referring to Scheme 5, instead of coupling a DOPA molecule (V) to a polymer (III) (e.g., Scheme 4), a polymeric or oligomeric chain of DOPA (VII) is coupled to a polymer (III). In some embodiments, the polymeric or oligomeric chain of DOPA (VII) can have 2 or more (e.g., 3 or more, 6 or more, 10 or more, 12 or more, or 15 or more) and/or 20 or less (e.g., 15 or less, 12 or less, 10 or less, 6 or less, or 3 or less) repeating units of DOPA (variable x). The polymeric or oligomeric DOPA (VII) can be made using standard peptide synthesis techniques, for example, by coupling monomeric DOPA with a coupling agent such as N,N′-dicyclohexylcarbodiimide.

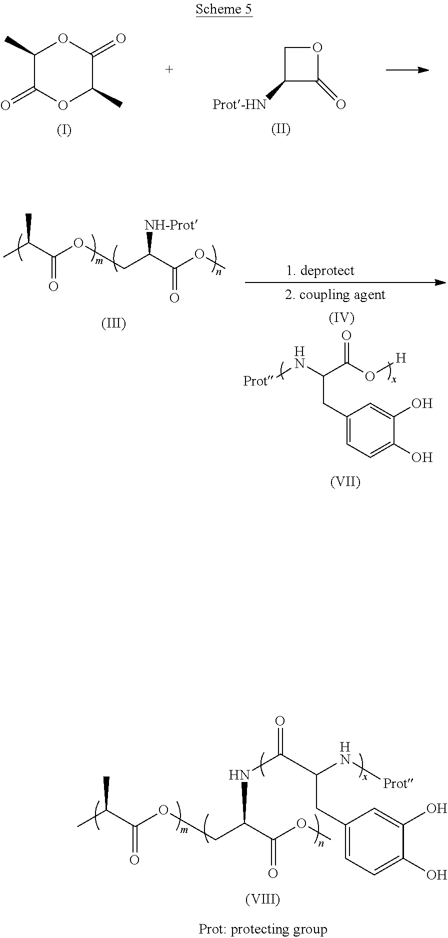

Referring to Scheme 6, in some embodiments, a protected functionalized polylactic acid (XI) is synthesized. The functionalized polylactic acid can then be deprotected (XII), coupled with a N-terminus protected DOPA (V) via an ester linkage, and finally deprotected to form a DOPA-functionalized poly(lactic acid) (XIV).

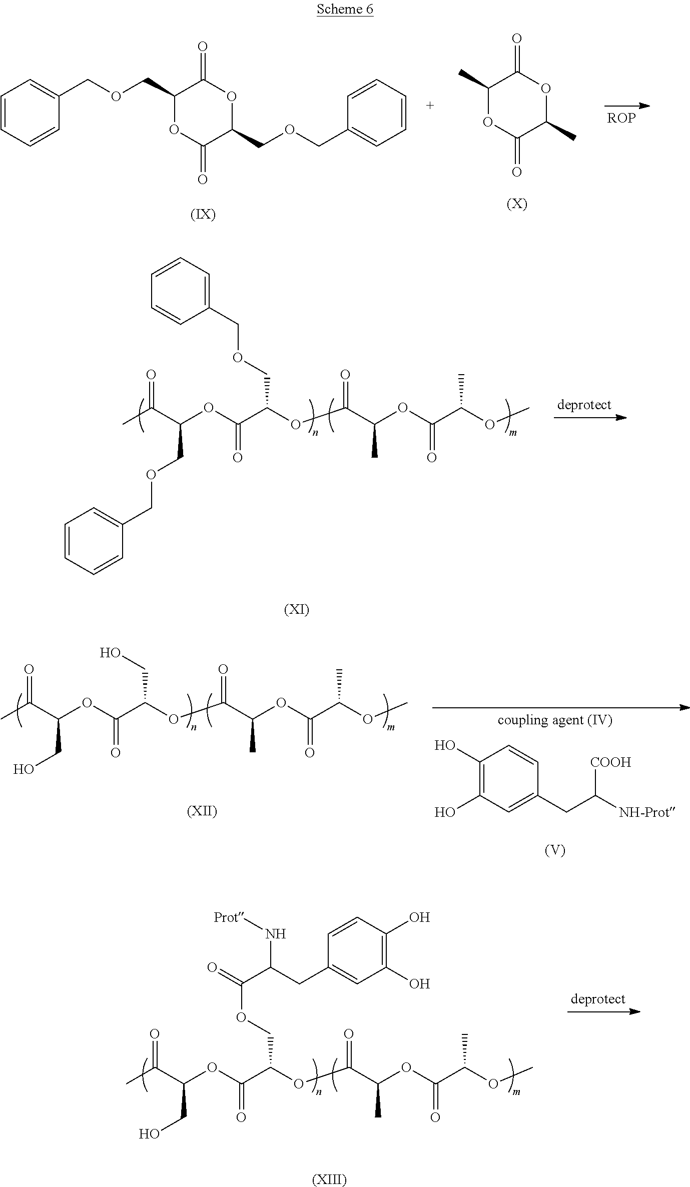

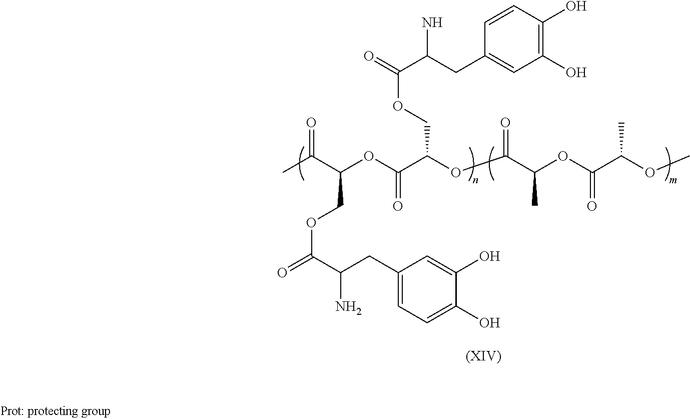

While the above schemes are provided for PLA polymers, the reaction schemes can be adapted to conjugate DOPA to other biodegradable polymers such as PLGA, PLLA, silk fibroin-chitosan conjugates, polysaccharides (including functionalized polysaccharides, e.g., chitosan, including functionalized chitosan), or blends thereof, using methods known to those of skill in the art. Silk fibroin-chitosan conjugates are described, for example, in Kang et al., Silk Fibroin/Chitosan Conjugate Crosslinked by Tyrosinase, Macromolecular Research, Vol. 12, No. 5, pp 534-539 (2004). Conjugation of DOPA to aminated polysaccharides can occur, for example, by reaction of a carbonyl moiety on quinone-DOPA with an amine from the aminated polysaccharide to form aza-type bonds. Further discussion of suitable synthesis techniques is provided in Nelson et al., “Protein-Bound 3,4-dihydroxy-phenylanine (DOPA), a Redox-Active Product of Protein Oxidation, as a Trigger for Antioxidant Defences”, (2007), 39 (5), 879-889; Kamber et al., “Organocatalytic Ring-Opening Polymerization”, Rev. (2007), 107, 5813-5840; Messersmith et al., U.S. Patent Application Publication No. 2003/0087338A1; Noga et al., “Synthesis and Modification of Functional Poly(lactide) Copolymers: Toward Biofunctional Materials”, Biomacromolecules (2008), 9, 2056-2062; Leemhuis et al., “Functionalized Poly(R-hydroxy acid)s via Ring-Opening Polymerization: Toward Hydrophilic Polyesters with Pendant Hydroxyl Groups”, Macromolecules (2006), 39, 3500-3508; Gerhardt et al., “Functional Lactide Monomers: Methodology and Polymerization”, Biomacromolecules (2006), 7, 1735-1742; Liu et al., “Convenient Synthesis of acetonide-protected 3,4-dihydroxyphenylalanine (DOPA) for Fmoc solid-phase peptide synthesis”, Tetrahedron Letters 49 (2008), 5519-5521; Silverman et al., “Understanding Marine Mussel Adhesion”, Marine Biotechnology Volume (2007), 9, 661-681; Lee et al., “Single-Molecule Mechanics of Mussel Adhesion”, PNAS, (2006), 103(35), 12999-13003; Wu L. et al., “Biofabrication: using biological materials and biocatalysts to construct nanostructured assemblies”, Trends in Biotechnology, (2004), 22(11), 593-599; and Yamada K. et al., “Chitosan Based Water-Resistant Adhesive. Analogy to Mussel Glue”, Biomacromolecules (2000), 1, 252-258.

### Stent Characteristics

A stent is bioerodible if the stent or a portion thereof exhibits substantial mass or density reduction or chemical transformation, after it is introduced into a patient, e.g., a human patient. Mass reduction can occur by, e.g., dissolution of the material that forms the stent and/or fragmenting of the stent. Chemical transformation can include oxidation/reduction, hydrolysis, substitution, and/or addition reactions, or other chemical reactions of the material from which the stent or a portion thereof is made. The erosion can be the result of a chemical and/or biological interaction of the stent with the body environment, e.g., the body itself or body fluids, into which it is implanted. The erosion can also be triggered by applying a triggering influence, such as a chemical reactant or energy to the stent, e.g., to increase a reaction rate. For example, a stent or a portion thereof can be formed from an active metal, e.g., Mg or Fe or an alloy thereof, and which can erode by reaction with water, producing the corresponding metal oxide and hydrogen gas; a stent or a portion thereof can also be formed from a bioerodible polymer, or a blend of bioerodible polymers which can erode by hydrolysis with water. Fragmentation of a stent occurs as, e.g., some regions of the stent erode more rapidly than other regions. The faster eroding regions become weakened by more quickly eroding through the body of the endoprosthesis and fragment from the slower eroding regions.

Preferably, the erosion occurs to a desirable extent in a time frame that can provide a therapeutic benefit. For example, the stent may exhibit substantial mass reduction after a period of time when a function of the stent, such as support of the lumen wall or drug delivery, is no longer needed or desirable. In certain applications, stents exhibit a mass reduction of about 10 percent or more, e.g. about 50 percent or more, after a period of implantation of about one day or more, about 60 days or more, about 180 days or more, about 600 days or more, or about 1000 days or less. Erosion rates can be adjusted to allow a stent to erode in a desired sequence by either reducing or increasing erosion rates. For example, regions can be treated to increase erosion rates by enhancing their chemical reactivity, e.g., coating portions of the stent with a silver coating to create a galvanic couple with the exposed, uncoated iron surfaces on other parts of the stent. Alternatively, regions can be treated to reduce erosion rates, e.g., by using protective coatings.

A coating can be deposited or applied over the surface of stent (e.g., over an entire surface, or over a part of a surface) to provide a desired function. The coating can include a DOPA-functionalized polymeric layer. In some embodiments, the coating includes a tie layer, a biocompatible outer coating, a radiopaque metal or alloy, and/or a drug-eluting layer.

A stent can include at least one releasable therapeutic agent, drug, or pharmaceutically active compound to inhibit restenosis, such as paclitaxel or everolimus, or to treat and/or inhibit pain, encrustation of the stent or sclerosing or necrosing of a treated lumen. As used herein, the terms “therapeutic agent”, “pharmaceutically active compound”, “pharmaceutically active agent”, “pharmaceutically active material”, “pharmaceutically active ingredient”, “drug” and other related terms may be used interchangeably and include, but are not limited to, small organic molecules, peptides, oligopeptides, proteins, nucleic acids, oligonucleotides, genetic therapeutic agents, non-genetic therapeutic agents, vectors for delivery of genetic therapeutic agents, cells, and therapeutic agents identified as candidates for vascular treatment regimens, for example, as agents that reduce or inhibit restenosis. By small organic molecule is meant an organic molecule having 50 or fewer carbon atoms, and fewer than 100 non-hydrogen atoms in total.

The therapeutic agent can be a genetic therapeutic agent, a non-genetic therapeutic agent, or cells. The therapeutic agent can also be nonionic, or anionic and/or cationic in nature. Exemplary therapeutic agents include, e.g., anti-thrombogenic agents (e.g., heparin); anti-proliferative/anti-mitotic agents (e.g., paclitaxel, 5-fluorouracil, cisplatin, vinblastine, vincristine, everolimus, inhibitors of smooth muscle cell proliferation (e.g., monoclonal antibodies), and thymidine kinase inhibitors); antioxidants; anti-inflammatory agents (e.g., dexamethasone, prednisolone, corticosterone); anesthetic agents (e.g., lidocaine, bupivacaine and ropivacaine); anti-coagulants; antibiotics (e.g., erythromycin, triclosan, cephalosporins, and aminoglycosides); agents that stimulate endothelial cell growth and/or attachment. Therapeutic agents can be used singularly, or in combination. Preferred therapeutic agents include inhibitors of restenosis (e.g., paclitaxel), anti-proliferative agents (e.g., cisplatin), and antibiotics (e.g., erythromycin). Examples of suitable therapeutic agents, drugs, or pharmaceutically active compounds include anti-thrombogenic agents, antioxidants, anti-inflammatory agents, anesthetic agents, anti-coagulants, and antibiotics, as described in U.S. Pat. No. 5,674,242; U.S. Ser. No. 09/895,415, filed Jul. 2, 2001; U.S. Ser. No. 11/111,509, filed Apr. 21, 2005; and U.S. Ser. No. 10/232,265, filed Aug. 30, 2002, the entire disclosure of each of which is herein incorporated by reference. Additional examples of therapeutic agents are described in U.S. Published Patent Application No. 2005/0216074.

The therapeutic agent, drug, or a pharmaceutically active compound can be incorporated in a polymeric coating carried by a stent. For example, in embodiments, the drug is incorporated within the porous regions in a polymer coating. Polymers for drug elution coatings are also disclosed in U.S. Published Patent Application No. 2005/019265A, the entire disclosure of which is herein incorporated by reference. A functional molecule, e.g., an organic, drug, polymer, protein, DNA, and similar material can be incorporated into grooves, pits, void spaces, and other features of the stent.

The polymeric materials described above can be used for the entire stent body, or a portion of the stent body or as a layer on a stent made of another material, or can include a layer of another material, which other material may be bioerodible or biostable, a metal, a polymer or a ceramic. In some embodiments, the stent can include one or more bioerodible metals, such as magnesium, zinc, iron, or alloys thereof. The stent can include bioerodible and non-bioerodible materials. The stent can have a surface including bioerodible metals, polymeric materials, or ceramics. The stent can have a surface including an oxide of a bioerodible metal.

In some embodiments, the stent can include one or more bioerodible metals, such as magnesium, zinc, iron, calcium, aluminum, or alloys thereof. The stent can include bioerodible and non-bioerodible materials. The stent can have a surface including bioerodible metals, polymeric materials, or ceramics. The stent can have a surface including an oxide of a bioerodible metal. Examples of bioerodible alloys also include magnesium alloys having, by weight, 50-98% magnesium, 0-40% lithium, 0-1% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Bioerodible magnesium alloys are also available under the names AZ91D, AM50A, and AE42. Other bioerodible alloys are described in Bolz, U.S. Pat. No. 6,287,332 (e.g., zinc-titanium alloy and sodium-magnesium alloys); Heublein, U.S. Patent Application 2002000406; and Park, Science and Technology of Advanced Materials, 2, 73-78 (2001), the entire disclosure of each of which is herein incorporated by reference. In particular, Park describes Mg—X—Ca alloys, e.g., Mg—Al—Si—Ca, Mg—Zn—Ca alloys. Examples of bioerodible polymers include polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), and combinations thereof.

A stent can also include non-bioerodible materials. Examples of suitable non-bioerodible materials include stainless steels, platinum enhanced stainless steels, cobalt-chromium alloys, nickel-titanium alloys, noble metals and combinations thereof. In some embodiments, stent 20 can include bioerodible and non-bioerodible portions. The stent can include (e.g., be manufactured from) metallic materials, e.g., biostable metallic materials such as stainless steel (e.g., 316L, BioDur® 108 (UNS S29108), and 304L stainless steel, and an alloy including stainless steel and 5-60% by weight of one or more radiopaque elements (e.g., Pt, Ir, Au, W) (PERSS®) as described in US-2003-0018380-A1, US-2002-0144757-A1, and US-2003-0077200-A1), Nitinol (a nickel-titanium alloy), cobalt alloys such as Elgiloy, L605 alloys, MP35N, titanium, titanium alloys (e.g., Ti-6A1-4V, Ti-50Ta, Ti-10Ir), platinum, platinum alloys, niobium, niobium alloys (e.g., Nb-1Zr) Co-28Cr-6Mo, tantalum, and tantalum alloys. Other examples of materials are described in commonly assigned U.S. application Ser. No. 10/672,891, filed Sep. 26, 2003; and U.S. application Ser. No. 11/035,316, filed Jan. 3, 2005. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned U.S. application Ser. No. 10/346,487, filed Jan. 17, 2003.

In some embodiments, non-bioerodible or biostable metals can be used to enhance the X-ray visibility of bioerodible stents. The bioerodible stent main structure of a stent can be combined with one or more biostable marker sections. The biostable marker sections can include, for example, gold, platinum or other high atomic weight elements. The biostable marker sections can provide enhance visibility and radiopacity and can provide a structural purpose as well. For example, any stent described herein can be dyed or rendered radiopaque by addition of, e.g., radiopaque materials such as barium sulfate, platinum or gold, or by coating with a radiopaque material.

A stent can have any desired shape and size (e.g., superficial femoral artery stents, coronary stents, aortic stents, peripheral vascular stents, gastrointestinal stents, urology stents, and neurology stents). Dependineon the application, stent 20 can have an expanded diameter of about 1 mm to about 46 mm. For example, a coronary stent can have an expanded diameter of about 2 mm to about 6 mm; a peripheral stent can have an expanded diameter of about 5 mm to about 24 mm; a gastrointestinal and/or urology stent can have an expanded diameter of about 6 mm to about 30 mm; a neurology stent can have an expanded diameter of about 1 mm to about 12 mm; and an abdominal aortic stent and a thoracic aortic stent can have an expanded diameter of about 20 mm to about 46 mm. Stent 20 can be self-expandable, balloon-expandable, or a combination of self-expandable and balloon-expandable (e.g., as described in U.S. Pat. No. 5,366,504). Stent 20 can have any suitable transverse cross-section, including circular and non-circular (e.g., polygonal such as square, hexagonal or octagonal).

A stent can be implemented using a catheter delivery system. Catheter systems are described in, for example, Wang U.S. Pat. No. 5,195,969; Hamlin U.S. Pat. No. 5,270,086; and Raeder-Devens, U.S. Pat. No. 6,726,712, the entire disclosure of each of which is herein incorporated by reference. Commercial examples of stents and stent delivery systems include Radius®, Symbiot® or Sentinol® system, available from Boston Scientific Scimed, Maple Grove, Minn.

A stent can be a part of a covered stent or a stent-graft. For example, a stent can include and/or be attached to a biocompatible, non-porous or semi-porous polymer matrix made of polytetrafluoroethylene (PTFE), expanded PTFE, polyethylene, urethane, or polypropylene. In addition to vascular lumens, a stent can be configured for non-vascular lumens. For example, it can be configured for use in the esophagus or the prostate. Other lumens include biliary lumens, hepatic lumens, pancreatic lumens, uretheral lumens and ureteral lumens.

All references, such as patent applications, publications, and patents referred to herein are incorporated by reference in their entirety.

Still further embodiments are in the following claims.

What is claimed is:

1. A stent, comprising:
a first layer including a biodegradable polymer functionalized with an adhesion enhancing amino acid.

2. The stent of claim 1, wherein the first polymer is on a stent body including stainless steel, ceramic, metal, metal alloys, metal oxides, metal nitrides, polymers, and combinations thereof.

3. The stent of claim 1, wherein the first layer is on a metal stent body.

4. The stent of claim 1, wherein the first layer is directly on a metal stent body.

5. The stent of claim 1, wherein the amino acid is DOPA.

6. The stent of claim 5, wherein DOPA is polymerized.

7. The stent of claim 1, wherein the polymer is PLA, PLGA, PLLA, polydioxanone, chitosan, polycaprolactone, blends thereof, or copolymers thereof.

8. The stent of claim 1, wherein the DOPA is oxidized.

9. The stent of claim 8, wherein the oxidized DOPA comprises a quinone or semiquinone moiety.

10. The stent of claim 1, wherein the DOPA is nonoxidized.

11. The stent of claim 2, wherein the stent further comprises a second layer over the first layer, the second layer including a second biodegradable polymer functionalized with an adhesion enhancing amino acid.

12. The stent of claim 11, further comprising a third layer over the first and second layers, wherein the third layer comprises a drug-eluting polymer.

13. The stent of claim 12, wherein the first or the second layer includes an oxidized DOPA.

14. The stent of claim 12, wherein the first or the second layer includes a nonoxidized DOPA.

15. The stent of claim 11, wherein the first and the second layers comprises, independently, a metal selected from the group consisting of iron, magnesium, and oxides thereof.

16. The stent of claim 11, wherein the stent body is stainless steel.

17. The stent of claim 4 wherein the first layer is on only the abluminal surface of the stent body.

18. The stent of claim 1 wherein the first layer further comprises a therapeutic agent.

19. The stent of claim 12, wherein one or more of the first layer, the second layer, and the third layer comprises a therapeutic agent.

20. A stent, comprising:
a stent body;
a first layer over the stent body comprising a biodegradable polymer functionalized with an adhesion enhancing amino acid;
a second layer over the first layer comprising a second biodegradable polymer functionalized with an adhesion enhancing amino acid; and
a third layer over the second layer comprising a drug-eluting polymer.

21. The stent of claim 20, wherein the amino acid is DOPA.
